(19) United States
(12) Patent Application Publication
(10) Pub. No.: US 2011/0196474 A1
(43) Pub. Date: Aug. 11, 2011

(54) MAGNETICALLY SENSITIVE DRUG CARRIERS FOR TREATMENT OR TARGETED DELIVERY

(75) Inventors: Dariush Davalian, San Jose, CA (US); Syed Faiyaz Ahmed Hossainy, Hayward, CA (US); Rachel Bright, Claremont, CA (US); Jinping Wan, San Jose, CA (US); Florian Niklas Ludwig, Mountain View, CA (US)

(73) Assignee: ABBOTT CARDIOVASCULAR SYSTEMS INC., Santa Clara, CA (US)

(21) Appl. No.: 12/704,136

(22) Filed: Feb. 11, 2010

(51) Int. Cl. A61F 2/82; A61K 47/02; A61K 9/14; A61K 9/127; A61P 35/00; A61P 9/10

(52) U.S. Cl. 623/1.15; 514/769; 424/489; 424/450

(57) ABSTRACT

Compositions of matter comprising a magnetically sensitive drug carrier and a related drug as well as methods for administering these compositions and causing them to localize within the patient using an internal or external magnetic field are described.

### BACKGROUND

Systemic delivery of drugs to a mammal is many millennia old if one considers medicinal herbs as drugs. When the overall ailment to be treated occur system wide, systemic delivery is a suitable delivery method. But in some cases of localized diseases, such as vascular or cardiovascular diseases, providing an effective concentration to the treated site using systemic delivery of the medication results in high drug concentrations throughout the patient. These high drug concentrations can produce adverse or toxic side effects. One the other hand, because in local delivery, the high concentrations are only local to the diseased site, local delivery can provide much lower systemic concentrations of medication throughout the patient. This concentration difference allows local delivery to cause fewer side effects and achieve better results. Unfortunately, local or regional delivery of a drug is much more difficult in most cases. What is needed is a delivery method that allows drug administration in a systemic manner, but also having the capability to act only or predominantly locally in the patient, thus keeping the system-wide drug concentration low while providing an effective concentration within the diseased region or at the diseased site.

### SUMMARY

According to an embodiment of the invention, a composition of matter comprising a drug and a magnetically sensitive drug carrier in which the composition is adapted for delivery to a patient and is also capable of responding to an internal or external magnetic field is described. In some embodiments, responding to a magnetic field means that particles of the composition experience a change in motion or a change in velocity when exposed to a magnetic field. In some embodiments, the composition is delivered to a cardiac or carotid artery related site.

In these or other embodiments, delivery is through a delivery pathway including a topical, enteral, or parenteral pathway. In these or other embodiments, the magnetically sensitive drug carrier is a nanoparticle, microparticle, liposome, micelle, nanofiber, or hydrogel. Some of these particles comprise ferrite particles, ferrous oxide, or rare earth particles. Some particles comprise a material that exhibits a ferromagnetic, superparamagnetic, or paramagnetic effect.

Furthermore, some embodiments described a method comprising administering the compositions described above to the patient and then applying an internal or external magnetic field or field gradient to the patient. In some embodiments, the magnetic field or field gradient causes the magnetically sensitive drug carrier (and carried drug) to localize within a region of the patient. Administering the composition can be accomplished through systemic, local, or semi-local means for various embodiments.

These or other embodiments, employ a delivery assistance technique, such as iontophoresis, electrophoresis, or sonophoresis.

Sometimes application of the magnetic field occurs after delivery of the composition. This time can range from 0.1 seconds to 365 days.

### DETAILED DESCRIPTION

The following description of several embodiments describes non-limiting examples that further illustrate the invention. All titles of sections contained herein, including those appearing above, are not to be construed as limitations on the invention, but rather they are provided to structure the illustrative description of the invention that is provided by the specification.

Unless defined otherwise, all technical and scientific terms used herein have the same meanings as commonly understood by one skilled in the art to which the disclosed invention pertains. The singular forms “a”, “an”, and “the” include plural referents unless the context clearly indicates otherwise.

While the description speaks in terms of a magnetically sensitive drug carrier, a drug, therapeutic substance, or bioactive agent molecule or agglomeration that itself has magnetic sensitivity, as described below, would fall within the scope of this description and claims. That is, this disclosure defines such a molecule as a magnetically sensitive drug carrier.

This invention discloses the use of magnetically sensitive drug carriers in conjunction with a magnetic field to target therapeutic agents to the carotid arteries, coronary arteries, superficial femoral arteries (FSA) such as femoral, popliteal or under the knee arteries such as posterior tibial, fibular, lateral cuneiform, medial plantar, medial digital, dorsal metatarsal, dorsal digital, dorsal common, dorsal pedis, arcuate, arcuate, anterior, tibial or other desired treatment region. Drug-loaded, magnetically sensitive carriers are delivered systemically. The delivery described in this document avoids the problems typically associated with systemic delivery by localizing the particles in or near the carotid arteries or other desired treatment region. To localize and retain these particles in the carotid arteries or other desired treatment region, a magnetic field is applied using any number of methods as discussed below. Once administered, these drug carriers release the drug over a preselected period at their localized site. In another embodiment, the drug-loaded magnetically sensitive carriers are delivered locally or interluminally while applying a magnetic field to the delivery site to avoid blood washing out the formulation.

Vulnerable plaque, diffuse atherosclerotic disease, aneurysm, anastomotic hyperplasia, chronic total occlusion, dysfunctional endothelium, recurring thrombus, fibrin accumulation or combination of these can be treated with the drug carriers described in this document.

Various embodiments of this invention provide a mechanism for efficient drug delivery to the arterial tree. For instance, a drug carrier is formulated to be responsive to an induced magnetic field. This formulation thus becomes a magnetically sensitive drug carrier and is applied to a biological system using systemic administration or local or regional administration, for example, to the pericardial sac. A magnetic field created by a device, for example, an intravascular catheter with a ferromagnet, will attract the formulated drug to the desired site to promote arterial loading of the formulated drug. This device may also be a permanent implant, such as an implanted magnet, or created from an external source, such as an external magnetic field. This would allow guidance of the magnetically sensitive drug carrier to the target site or region, and further may allow increased or controlled arterial concentrations, region-specific delivery, or time-controlled delivery of the magnetically sensitive drug carrier. The field could be a fluctuating field to enhance penetration of the particles. The drug would then be released from the formulation to influence a selected biological process. This magnetic field may be applied rapidly after administration of the magnetically sensitive drug carrier, or may occur later, or at multiple times. Some embodiments are particularly useful in areas with shallow arteries, such as peripheral arteries. Peripheral arteries occur in the leg, knee, and below-knee regions, among other regions. A magnet can be attached to the skin or worn around the leg, knee, or below the knee to increase the residence time of the particles for better penetration.

In one embodiment, the invention is a composition of matter that comprises the drug in the magnetically sensitive drug carrier. This composition is adapted for delivery to a mammal and, since it contains a magnetically sensitive drug carrier, the composition is capable of responding to magnetic field. In some embodiments adapted for delivery to a mammal means adapted for delivery to a mammalian cardiac-related site or carotidartery-related site such as those sites within the human patient. In some embodiments, delivery to a cardiac-related site is delivery to the heart.

### Magnetically Sensitive Drug Carrier

In various embodiments, magnetically sensitive drug carriers can be nanoparticles or microparticles, liposomes, micelles, nanofibers, hydrogel, or the like. The magnetic sensitivity can reside in the base material of the particle or a separate material with magnetic sensitivity can be added to the particle during or after the particle's manufacture. Depending upon the delivery method, the particles of the magnetically sensitive drug carriers can range in size from 10 nm to 2000 nm or 20 nm to 300 nm.

Likewise, the particles of the magnetically sensitive drug carriers can range in size from 1 nanometer for ferrous compound particles to several microns for some liposomes.

### Magnetic Particles and Beads

Magnetic particles and beads sourced from or made similarly to beads sourced from the companies listed below are useful in the practice of the current invention.
Sera-Mag™ magnetic particles are based on U.S. Pat. No. 5,648,124, and use 1 μM magnetic carboxylate-modified base particles made by a core-shell process. The entire contents of U.S. Pat. No. 5,648,124 are hereby incorporated by this reference;
Iron-containing nanoparticles available from Ocean Nanotech;
Functionalized magnetic beads such as those available from Bioclone, Inc.;
Coated magnetic particles such as those available from Spherotech; and
Coated magnetic beads such as those available from ThermoScientific.

### Liposomes

Liposomes available from Encapsula Nano Sciences are also useful as the magnetically sensitive drug carrier. Other liposomes useful in the practice of this invention can be made by methods disclosed in the following references:
Bimodal Paramagnetic and Fluorescent Liposomes for Cellular and Tumor Magnetic Resonance Imaging; Kamaly, Nazila; Kalber, Tammy; Ahmad, Ayesha; Oliver, Morag H.; So, Po-Wah; Herlihy, Amy H.; Bell, Jimmy D.; Jorgensen, Michael R.; Miller, Andrew D.; Imperial College Genetic Therapies Centre, Department of Chemistry, Imperial College London, London, UK; Bioconjugate Chemistry (2008), 19(1), 118-129;
Preparation and characterization of novel magnetic cationic polymeric liposomes. Liang, Xiao-Fei; Wang, Han-Jie; Tian, Hui; Luo, Hao; Cheng, Jing; Hao, Li-Juan; Chang, Jin. Institute of Nanobiotechnology, School of Materials Science and Engineering, Tianjin University, Tianjin, Peop. Rep. China. Gaodeng Xuexiao Huaxue Xuebao (2008), 29(4), 858-861;
The effect of magnetic targeting on the uptake of magnetic-fluid-loaded liposomes by human prostatic adenocarcinoma cells. Martina, Marie-Sophie; Wilhelm, Claire; Lesieur, Sylviane. Equipe Physico-Chimie des Systemes Polyphases, CNRS UMR 8612, Chatenay, Malabry, Fr. Biomaterials (2008), 29(30), 4137-4145;
Preparation and use of magnetically guided liposomes in treatment of oncological diseases. Alyautdin, R. N.; Torshina, N. L.; Cherkasova, O. G.; Filippov, V. I.; Larin, M. Yu.; Ivanov, P. K.; Blokhin, D. Yu.; Bayburtskiy, F. S. I. M. Sechenov Medical Academy, Moscow, Russia. Oxidation Communications (2006), 29(4), 924-931;
Biotechnology of Magnet-Driven Liposome Preparations. Ismailova, G. K.; Efremenko, V. I.; Kuregyan, A. G. State Pharmaceutical Academy, Pyatigorsk, Russia. Pharmaceutical Chemistry Journal (2005), 39(7), 385-387; and
Injectable magnetic liposomes as a novel carrier of recombinant human BMP-2 for bone formation in a rat bone-defect model. Matsuo, Toshihiro; Sugita, Takashi; Kubo, Tadahiko; Yasunaga, Yuji; Ochi, Mitsuo; Murakami, Teruo. Department of Orthopaedic Surgery, Programs for Applied Biomedicine, Division of Clinical Medical Science, Graduate School of Biomedical Sciences, Hiroshima University, Hiroshima, Japan. Journal of Biomedical Materials Research, Part A (2003), 66A(4), 747-754.

### Micelles

Micelles useful in the practice of this invention can be made by methods disclosed in the following references:
Synthesis and surface engineering of superparamagnetic iron oxide nanoparticles for drug delivery and cellular targeting. Gupta, Ajay Kumar; Gupta, Mona. Formulation Development Department, Torrent Research Centre, Torrent Pharmaceutical Limited, Gujarat, India. Editor(s): Kumar, M. N. V. Ravi. Handbook of Particulate Drug Delivery (2008), 1 205-221.
Micellar hybrid nanoparticles for simultaneous magnetofluorescent imaging and drug delivery. Park, Ji-Ho; von Maltzahn, Geoffrey; Ruoslahti, Erkki; Bhatia, Sangeeta N.; Sailor, Michael J. Materials Science and Engineering Program, Department of Chemistry and Biochemistry, University of California, San Diego, La Jolla, Calif., USA. Angewandte Chemie, International Edition (2008), 47(38), 7284-7288.
Preparation and characterization of PNIPAAm-bPLA/Fe₃O₄ thermo-responsive and magnetic composite micelles. Ren, Jie; Jia, Menghong; Ren, Tianbin; Yuan, Weizhong; Tan, Qinggang. Institute of Nano and Bio-Polymeric Materials, School of Material Science and Engineering, Tongji University, Shanghai, Peop. Rep. China. Materials Letters (2008), 62(29), 4425-4427.
cRGD-encoded, MRI-visible polymeric micelles for tumor-targeted drug delivery. Gao, Jinming; Nasongkla, Norased; Khemtong, Chalermchai. Simmons Comprehensive Cancer Center, University of Texas Southwestern Medical Center, Dallas, Tex., USA. Editor(s): Amiji, Mansoor M. Nanotechnology for Cancer Therapy (2007), 465-475;
Diacyllipid Micelle-Based Nanocarrier for Magnetically Guided Delivery of Drugs in Photodynamic Therapy. Cinteza, Ludmila O.; Ohulchanskyy, Tymish Y.; Sahoo, Yudhisthira; Bergey, Earl J.; Pandey, Ravindra K.; Prasad, Paras N. Institute for Lasers Photonics and Biophotonics, SUNY at Buffalo, Buffalo, N.Y., USA. Molecular Pharmaceutics (2006), 3(4), 415-423;
Synthesis of magnetic nanoparticles and their application to bioassays. Osaka, Tetsuya; Matsunaga, Tadashi; Nakanishi, Takuya; Arakaki, Atsushi; Niwa, Daisuke; lida, Hironori. Department of Applied Chemistry, Waseda University, 3-4-1 Okubo, Shinjuku, Japan. Analytical and Bioanalytical Chemistry (2006), 384(3), 593-600;
Magnetite-loaded polymeric micelles as ultrasensitive magnetic-resonance probes. Ai, Hua; Flask, Christopher; Weinberg, Brent; Shuai, Xintao; Pagel, Marty D.; Farrell, David; Duerk, Jeffrey; Gao, Jinming. Department of Biomedical Engineering Case Western, Reserve University, Cleveland, Ohio, USA. Advanced Materials (Weinheim, Germany) (2005), 17(16), 1949-1952; and
Reverse Micelle Synthesis and Characterization of Superparamagnetic MnFe₂O₄Spinel Ferrite Nanocrystallites. Liu, Chao; Zou, Bingsuo; Rondinone, Adam J.; Zhang, Z. John. School of Chemistry & Biochemistry, Georgia Institute of Technology, Atlanta, Ga., USA. Journal of Physical Chemistry B (2000), 104(6), 1141-1145.

### Hydrogel

Hydrogels useful in the practice of this invention can be made by methods disclosed in the following references:
Study on controlled drug permeation of magnetic-sensitive ferrogels: Effect of Fe₃O₄ and PVA. Liu, Ting-Yu; Hu, Shang-Hsiu; Liu, Kun-Ho; Liu, Dean-Mo; Chen, San-Yuan. Department of Materials Sciences and Engineering, National Chiao Tung University, Hsinchu, Taiwan. Journal of Controlled Release (2008), 126(3), 228-236;
Controlled Pulsatile Drug Release from a Ferrogel by a High-Frequency Magnetic Field. Hu, Shang-Hsiu; Liu, Ting-Yu; Liu, Dean-Mo; Chen, San-Yuan. Department of Materials Sciences and Engineering, National Chiao Tung University, Hsinchu, Taiwan. Macromolecules (Washington, D.C., United States) (2007), 40(19), 6786-6788;
Composites of polymeric gels and magnetic nanoparticles: preparation and drug release behavior. Francois, Nora J.; Allo, Sabina; Jacobo, Silvia E.; Daraio, Marta E. Laboratorio de Aplicaciones de Polimeros Hidrofilicos, Departamento de Quimica, Facultad de Ingenieria, Universidad de Buenos Aires, Buenos Aires, Argent. Journal of Applied Polymer Science (2007), 105(2), 647-655;
Synthesis and temperature response analysis of magnetic-hydrogel nanocomposites. Frimpong, Reynolds A.; Fraser, Stew; Hilt, J. Zach. Department of Chemical and Materials Engineering, University of Kentucky, Lexington, Ky., USA. Journal of Biomedical Materials Research, Part A (2006), Volume Date 2007, 80A(1), 1-6;
PVP magnetic nanospheres: Biocompatibility, in vitro and in vivo bleomycin release. Ding, Guowei; Adriane, Kamulegeya; Chen, XingZai; Chen, Jie; Liu, Yinfeng. Tongji university hospital, Shanghai, Peop. Rep. China. International Journal of Pharmaceutics (2007), 328(1), 78-85; and
Preparation and characterization of magnetic targeted drug controlled-release hydrogel microspheres. Chen, Jie; Yang, Liming; Liu, Yinfeng; Ding, Guowei; Pei, Yong; Li, Jian; Hua, Guofei; Huang, Jian. Department of Chemical Engineering and Technology, Shanghai University, Shanghai, Peop. Rep. China. Macromolecular Symposia (2005), 225(Polymers in Novel Applications), 71-80.

### Polymeric Nanoparticles

Polymeric Nanoparticles useful in the practice of this invention can be made by methods disclosed in the following references:
Seung-Jun Lee et al. Journal of Magnetism and Magnetic Materials 272-276 (2004) 2432-2433;
S. A. Gomez-Lopera et al. Journal of Colloid and Interface Science 240, 40-47 (2001);
L. Ngaboni Okassa et al. International Journal of Pharmaceutics 302 (2005) 187-196;
Seung-Jun Lee et al. Colloids and Surfaces A: Physicochem. Eng. Aspects 255 (2005) 19-25; and
Bryan R. Smith et al. Biomed Microdevice (2007) 9:719-727.

Drug-loaded particles comprise a magnetically sensitive component such as ferrite particles, ferrous oxide, rare earth particles, and the like. Particles may comprise polymer, degradable polymer, biodegradable glass or biodegradable metal, lipids, and the like.

In some embodiments, the magnetic agents are encapsulated into the nanoparticles or other carriers during the encapsulation process (e.g. emulsion, spray drying, and electrospraying, etc.) without interacting with the drugs or destroying the magnetic character of the magnetic agent.

In some embodiments, the magnetically sensitive drug carrier may comprise an oxidizing agent. The particle size for some embodiments of this magnetically sensitive drug carrier would be <1 micron, and preferably <500 nm, to increase the ability of the particle to migrate through the tissue. This particle would be delivered into the pericardial sac with the use of a surgical technique, or using an intravascular approach, delivered to create a reservoir of a magnetically sensitive drug carrier comprising, as the drug component, an antioxidant. Subsequently, at a desired time, such as following a myocardial infarction, a catheter could be introduced into the coronary tree, and positioned in a region of affected ischemic tissue, near the infarction site. A magnetic field generated from this device would draw the particles to the arterial site. At this arterial site, they would deliver the anti-oxidant to influence infarct progression.

### Methods of Associating Magnetically Sensitive Drug Carrier with the Drug or Drugs

The drug or drugs can be attached to or contained in the magnetically sensitive carrier in a variety of ways. In various embodiments, the drug is within the particle (internal to the particle), located within pores in the particle (for porous particles), absorbed to the surface of the particle, conjugated to the surface of the particle, or simply mixed with the particle material.

In some embodiments, the magnetic nanoparticles (such as metal oxide particles) conjugate with the therapeutic agents through a cleavable linker. The linker's design allows it to release the drug component by acid hydrolysis, reduction, oxidation, or photochemical or enzymatic action either present in the tissue or induced externally. The linker is an assembly of atoms attached to one another, in some embodiments, through chemical bonds. The linker, in some embodiments, attaches to the at least two parts of the magnetically sensitive drug carrier: the drug part and the magnetically sensitive carrier part. In some embodiments, the attachment occurs through chemical bonds. In some of these or other embodiments, the chemical bonds are covalent.

### Drug Transfer from Particle to Tissue

Once the magnetically sensitive drug carrier has been localized by application of the magnetic field, the drug should leave the particle and enter the tissue or diseased tissue at the treatment site. For particles in which the drug is absorbed into or onto the particle, this “leaving” can be by diffusion. In some embodiments, diffusion may be the rate-limiting step. For particles in which the drug is absorbed into pores in the particle, this “leaving” can be by diffusion out of the pores. For particles in which the drug is attached, such as through a chemical bond directly to the drug or through a set of linking atoms, this “leaving” can be by breaking the chemical bond between the drug and the particle. In some embodiments, the rate-limiting step, after localizing the magnetically sensitive particles, in the process of the drug moving from a particle to the tissue is the breaking of the chemical bond or connection between the particles and the drug. In some embodiments, the drug may be able to act on the tissue without “leaving” the particle.

In some embodiments, The conjugated drugs will be directed to the target site by the magnetic field and will release the drug over time.

Different varieties of magnetically sensitive drug carriers and different delivery method or pathways are discussed below.

Various embodiments of this invention are useful for the treatment of vascular dysfunction in which local delivery of a drug, in a controlled or reoccurring manner, would be beneficial, such as chronic arterial disease. This invention is used for treating any locally manifesting disease in which controlled dosing of a drug at a specific location would be beneficial.

Additionally, this invention may also be used to treat other vessels or tissue, including cancer located close to the vascular surface or having appropriate vascular access.

### Generation of Magnetic Field

The magnetically sensitive drug carrier will be attracted to the delivery site with the use of a magnetic field created by a device, for example, by an intravascular catheter device with a ferromagnet, to promote arterial loading of the drug. This device may also be a permanent implant, such as an implanted magnet, or an external magnet or magnetic field. The field could be a fluctuating field to enhance penetration of the particles.

For purposes of this disclosure, magnetic field means (1) a magnetic field with its accompanying field gradient caused by the natural decrease in field strength as the distance between the source and the magnetic material increases; (2) an engineered magnetic field gradient that is purposely constructed, such as with an electromagnetic solenoid or a permanent or electromagnet with poles shaped to provide the desired gradient; or (3) a combination of (1) and (2).

The magnetic fields can be from one or more permanent magnets or from electromagnets. These localized field sources can be outside the patient, inside the patient, or a combination of both. External fields have the advantage of being easier and more convenient to apply to the patient. On the other hand, since a magnet's field strength diminishes rapidly as the distance from the magnet to the target increases, external magnetic field sources need to be much more intense than internal magnetic field sources. The shape of the magnet greatly affects the resulting field. This allows tailoring of the field or field shape to the desired particle localization method. For instance, properly shaped electromagnets or permanent magnets could cause a large magnetic field or large magnetic field gradient to center on the area to be treated, such as the heart or cardiovascular system. Similarly, using an electromagnet, the magnetic field can be turned on and off or otherwise pulsed, for instance between two different field strengths. (This would help the particle to penetrate the tissue or embed in the tissue better).

### Magnetic Materials

A magnetically sensitive drug carrier requires enough magnetic material to be sensitive to or to respond to the magnetic field. For purposes of this disclosure, respond means that the magnetic field is capable of causing a change in the motion of the magnetically sensitive drug carrier particles. Thus, one of ordinary skill in the art appreciates that enough magnetic material depends, in part, on the size of the particle, the magnitude or shape of the magnetic field, the distance to the magnetic field, or the magnetic strength of the magnetic material (otherwise known as the magnetization M).

In some embodiments, respond to the magnetic field means that the drug carrier experiences a change in motion (due to the magnetic field) such that drug delivery is improved in any way over the same drug carrier absent the magnetic field source. In some embodiments, response of magnetic field means that the particles are directed to the desired treatment area long enough to improve or increase the drug transfer from the drug carrier to the tissue versus the drug carrier in the absence of the magnetic field. Beneficial changes in any of the following parameters can be used as indices of efficacy. In some cases, the parameters can be classified as parameters related to tissue composition, such as lipid composition, inflammation, apoptosis, fibrosis etc. Alternatively, the parameters can be classified as related to function such as changes in blood flow, oxygenation, electrophysiology etc.

Various places throughout this disclosure refer to an improved drug delivery or a drug delivery that is improved in any way. Improvement or improved in any way means that the drug delivery or drug transfer is quantitatively or qualitatively improved in any way that one of ordinary skill in the art would recognize as being somehow better than a non-improved drug delivery technique. A subset of these art-recognized improvements includes an improvement in tissue concentration of the drug in the target area, an improvement in the tissue concentration of the drug in peripheral tissue without any tissue-drug-concentration degradation in the target tissue, regression or non-progression of the disease in diseased tissue, increased regression of the disease in the diseased tissue when using magnetically sensitive drug carriers than when using drug carriers that are not magnetically sensitive, stabilization or improvement in the patient's condition or increased improvement in the patient's condition using magnetically sensitive drug carriers over the improvements seen in patients using drug carriers that are not magnetically sensitive.

The extent of drug delivery or drug transfer is typically measured by measuring the drug content of the target tissue. Therefore, drug delivery improvements cause a change in drug delivery that results in the measured drug content becoming closer to the clinically required or desired amount. Therefore, any change in the magnetically sensitive drug carrier's motion over that of similar drug carriers, lacking substantial magnetic sensitivity, that brings about a tissue-drug content that is closer to the desired amount is an improvement.

Since one of the goals of local or regional treatment is to minimize the unwanted effects of drug on peripheral tissue as a target tissue is dosed, any change in drug delivery that causes less harm or drug exposure to peripheral tissue can be called an improvement. But to be an improvement, it must keep drug delivery unchanged or at least retain adequate drug delivery to the target tissue.

Improvement in a more qualitative sense can be an improvement in the health of the diseased tissue brought about by using magnetically sensitive drug carriers as opposed to drug delivery with similar, but not magnetically sensitive, drug carriers. Alternatively, improvement in the health of the diseased tissue using magnetically sensitive drug carriers as compared to no treatment at all is another qualitative way of determining or measuring the improvement brought about by using magnetically sensitive drug carriers.

Another qualitative measurement of improvement of drug delivery is an improvement in the patient's condition after treatment with a drug carried by the magnetically sensitive drug carriers versus treatment using a drug carrier that does not have magnetically sensitive components. Alternatively, improvement in the patient's condition after treatment with the drug carried by magnetically sensitive drug carrier as opposed to no treatment at all is another qualitative way of determining or measuring the improvement brought about by using magnetically sensitive drug carriers.

Response to the magnetic field can be characterized in other ways, as well. Usually, the amount (concentration) of drug in the target tissues has units like, nanograms of drug per gram of tissue. There is usually a minimum effective dose of the drug in question. Thus, in some embodiments, to respond to the magnetic field means that the particles are kept within the desired treatment area long enough to allow drug transfer significant enough that the concentration of the drug in the target tissues rises enough above the minimum effective dose to be therapeutically significant. Alternatively, to respond to the magnetic field means that the particles reside within the desired treatment area long enough to allow drug transfer significant enough that the time that the target tissue drug concentration is above the minimum effective dose is therapeutically significant. Therapeutically significant usually means that the therapy provides a detectable improvement in an objective measurement of a disease parameter (like restenosis rate, vessel ID, ejection fraction, etc.) or a detectable slowing of progression in the disease symptoms (like angina, walking distance, and CHF class) or lowered death rates.

A useful magnetic material in the magnetically sensitive drug carrier is a ferromagnetic material. Ferromagnetic materials are materials that have permanent magnetic moments, hence magnetism on a macroscopic scale. Ferromagnetic materials have magnetic domains that each have a magnetic moment simplistically made up of the contributions of the unpaired electrons on the atoms (or in some cases, molecules) of the material. In the absence of thermal energy in the ferromagnetic material, all of the magnetic moments of the magnetic domains would align. But at room temperature, for instance, the thermal energy causes misalignment between the magnetic moments of the domains. Nonetheless, at least some residual alignment remains yielding magnetism in the material.

Thus, ferromagnetic materials are useful for inclusion in the magnetically sensitive drug carriers described in this disclosure, if they have the other chemical properties necessary to be safe for use in pharmaceutical compositions. Ordinarily skilled artisans know these properties well.

Moreover, paramagnetic and super-paramagnetic materials could be used as the magnetic material for the magnetically sensitive drug carriers described in this disclosure. Since these materials do not have a permanent magnetic moment at treatment temperatures, their use as a magnetic component of the magnetically sensitive drug carrier requires two magnetic fields or at least one field gradient. One of these magnetic fields causes the magnetic moments in the materials to align; the other magnetic field causes the localization (as this term is used in the current disclosure) of the aligned paramagnetic atoms or molecules contained in the magnetically sensitive drug carriers.

In some embodiments, a core of magnetically sensitive particles comprises magnetite particles (such as those made from FeCl₃ and FeCl₂) and stabilized with fatty acids such as oleic acid, to give hydrophobic properties to the magnetite. These particles then can be incorporated into polymeric nanoparticles, liposomes, or micelles by methods known in the art.

Specific compositions of useful magnetically sensitive components of the magnetically sensitive drug particles include certain elements and compounds. Elements can be paramagnetic if they have unpaired electrons. The following are some examples of paramagnetic elements:
Aluminum (metal)
Barium (metal)
Oxygen (non-metal)
Platinum (metal)
Sodium (metal)
Strontium (metal)
Uranium (metal)
Technetium (metal)
Dysprosium (metal)—ferromagnetic

Many salts or compounds of the d and f transitional metal group show paramagnetic behavior. The following are some examples of paramagnetic compounds:
Copper sulphate
Dysprosium oxide
Ferric chloride
Ferric oxide
Holmium oxide
Manganese chloride

The force exerted on magnetically responsive particles is proportional to the gradient of the magnetic field and the magnetic moment of the particle. In cases where the magnetic moment is induced, e.g. in the case of paramagnetic or superparamagnetic particles, the particle magnetic moment, and therefore the force exerted on it, also becomes a function of the magnitude of the external magnetic field.

### Delivery Routes or Pathways

Delivery routes for the compositions described in this disclosure depend somewhat on the composition's particle size. One of ordinary skill in the art can select the composition's particle size to tailor its suitability for many different delivery pathways or delivery routes including topical, enteral, and parenteral pathways or delivery routes, among other routes.

In principle, the magnetically sensitive drug carrier is delivered through a pathway either systemically or semi-systemically providing carrier particles throughout the vasculature in the case of arterial or venous delivery or throughout the organ's vasculature in the case of delivery near an organ. Also, for cardiac treatment, the carrier particles can be delivered to the pericardial sac surrounding the heart.

Once delivered, a magnetic field is used to interact with the particles. In some embodiments, such interaction is sufficient to localize the particles. Localize, for the purpose of this disclosure, means slowing migration of the particles through the treatment locale enough that the drug can diffuse to the tissue in question more effectively than if no magnetic field were applied. In some embodiments, such as those involved with vascular treatment, localize means that the particles are slowed such that they have 50%, 40%, 30%, 20%, 10%, 5%, or 1% of the average velocity of the blood cells in that locale.

In some embodiments, localize means that the particles are stopped or caused to deposit near the treatment location. In some embodiments, the magnetic field causes the magnetically sensitive drug carrier particles to embed in the tissue near the treatment site.

For purposes of this disclosure, topical delivery means having local effect: the substance is applied directly where its action is desired. Examples of topical delivery include epicutaneous (application onto the skin), inhalational, enema, eye drops (onto the conjunctiva), eardrops, intranasal route (into the nose), and vaginal delivery or pathways.

For purposes of this disclosure, enteral means delivery is systemic (non-local) and involves part of the gastrointestinal tract. Examples of enteral delivery include by oral, by gastric feeding tube, by duodenal feeding tube, by gastrostomy, or by rectal delivery or pathways.

For purposes of this disclosure, parenteral delivery means delivery is systemic and the substance is given by routes other than the digestive tract. Examples of parenteral delivery include intravenous, intra-arterial, intramuscular, intracardial, subcutaneous (under the skin), intraosseous infusion (into the bone marrow), intradermal (into the skin itself), intrathecal (into the spinal canal), intraperitoneal (infusion or injection into the peritoneum), intravesical infusion (infusion into the urinary bladder), transdermal (diffusion through the intact skin), transmucosal (diffusion through a mucous membrane), sublingual, buccal (absorbed through cheek near gum line), inhalational, epidural (injection or infusion into the epidural space), and intravitreal (through the eye).

Drug-loaded, magnetically sensitive carriers may be applied topically with a formulation that enhances penetration through the skin. The skin penetration can be assisted by iontophoresis, electrophoresis, or sonophoresis.

### Therapeutic Substances

For any of the foregoing embodiments that contain or deliver drugs including from stents or from balloons such as angioplasty balloons adapted for drug delivery or drug delivery balloons can use a drug or therapeutic substance selected from those described in this section. Generally, this disclosure uses the term “drug” and “therapeutic substance” interchangeably throughout.

Therapeutic substances are biologically active agents. Therapeutic substances can be, for example, therapeutic, prophylactic, or diagnostic agents. As used in this document, the therapeutic substance includes a bioactive moiety, derivative, or metabolite of the therapeutic substance.

Examples of suitable therapeutic and prophylactic agents include synthetic inorganic and organic compounds, proteins and peptides, polysaccharides and other sugars, lipids, and DNA and RNA nucleic acid sequences having therapeutic, prophylactic, or diagnostic activities. Nucleic acid sequences include genes, antisense molecules, which bind to complementary DNA to inhibit transcription, and ribozymes. Other examples of therapeutic substances include antibodies, receptor ligands, and enzymes, adhesion peptides, oligosaccharides, blood clotting factors, inhibitors or clot dissolving agents, such as streptokinase and tissue plasminogen activator, antigens for immunization, hormones and growth factors, oligonucleotides such as antisense oligonucleotides and ribozymes and retroviral vectors for use in gene therapy,

In other examples, the drugs or therapeutic substances inhibit vascular-smooth-muscle-cell activity. More specifically, the therapeutic substance may inhibit abnormal or inappropriate migration or proliferation of smooth muscle cells leading to restenosis inhibition. Therapeutic substances can also include any substance capable of exerting a therapeutic or prophylactic effect in the practice of the present invention. For example, the therapeutic substance could be a prohealing drug that imparts a benign neointimal response characterized by controlled proliferation of smooth muscle cells and controlled deposition of extracellular matrix with complete luminal coverage by phenotypically functional (similar to uninjured, healthy intima) and morphologically normal (similar to uninjured, healthy intima) endothelial cells.

The therapeutic substance can also fall under the genus of antineoplastic, cytostatic or anti-proliferative, anti-inflammatory, antiplatelet, anticoagulant, antifibrin, antithrombin, antimitotic, antibiotic, antiallergic and antioxidant substances.

Antineoplastic or antimitotic examples:
paclitaxel
docetaxel
methotrexate
Azathioprine
Vincristine
Vinblastine
Fluorouracil
doxorubicin hydrochloride
mitomycin

Antiplatelet, anticoagulant, antifibrin, and antithrombin examples:
Heparinoids
Hirudin
Argatroban
Forskolin
Vapiprost
Prostacyclin
prostacyclin analogues
Dextran
D-phe-pro-arg-chloromethylketone (synthetic antithrombin)
Dipyridamole
glycoprotein IIb/IIIa platelet membrane receptor antagonist antibody
recombinant hirudin and thrombin inhibitors

Cytostatic or Antiproliferative Agent Examples
Angiopeptin
angiotensin converting enzyme inhibitors
cilazapril
lisinopril
actinomycin D
dactinomycin
actinomycin IV
actinomycin
actinomycin X₁
actinomycin
actinomycin D derivatives or analogs

Other therapeutic substances include
calcium channel blockers
nifedipine
Colchicines
fibroblast growth factor (FGF) antagonists
omega 3-fatty acid
Fish oil
Flax seed oil
histamine antagonists
lovastatin
monoclonal antibodies (such as those specific for
Platelet-Derived Growth Factor (PDGF) receptors)
Nitroprusside
phosphodiesterase inhibitors
prostaglandin inhibitors
Suramin
serotonin blockers
Steroids
thioprotease inhibitors
triazolopyrimidine (a PDGF antagonist)
nitric oxide
alpha-interferon
genetically engineered epithelial cells
antibodies such as CD-34 antibody
abciximab (REOPRO)
progenitor cell capturing antibody
pro-healing therapeutic substances (that promotes controlled proliferation of muscle cells with a normal and physiologically benign composition and synthesis product)
Enzymes
anti-inflammatory agents
Antivirals
anticancer drugs
anticoagulant agents
free radical scavengers
Estradiol
steroidal anti-inflammatory agents
non-steroidal anti-inflammatory
dexamethasone
clobetasol
aspirin
Antibiotics
nitric oxide donors
Photosensitizers for photodynamic therapy
SiRNAs
super oxide dismutases
super oxide dismutase mimics
4-amino-2,2,6,6-tetramethylpiperidine-1-oxyl (4-amino-TEMPO)
Tacrolimus
Rapamycin
rapamycin derivatives 40-O-(2-hydroxy)ethyl-rapamycin (everolimus)
40-O-(3-hydroxy)propyl-rapamycin
40-O-[2-(2-hydroxy)ethoxy]ethyl-rapamycin
40-O-tetrazole-rapamycin
Zotarolimus™
cytostatic agents

An example of an antiallergic agent is permirolast potassium.

The foregoing substances are listed by way of example and are not meant to be limiting. Other active agents that are currently available or that may be developed in the future are equally applicable.

In at least one embodiment, the invention is a composition of matter comprising a drug and a magnetically sensitive drug carrier, such as a nanoparticle, microparticle, liposome, micelle, nanofiber, or hydrogel, wherein the magnetically sensitive drug carrier comprises ferrite particles, ferrous oxide, or rare earth particles, and wherein the composition is adapted for delivery to a mammal and capable of responding to a magnetic field.

### EXAMPLES

### Example 1

### Preparation of Hydrophobic Magnetite

The coprecipitation method of preparing gelatinous hydrophobic magnetite was previously reported. Briefly, FeCl₃.6H₂O (23.2 g) and FeCl₂.4H₂O (8.60 g) would be dissolved in 800 ml of deionized water under nitrogen atmosphere with vigorous stirring at 90 C. Ammonium hydroxyide solution (15 ml) would be slowly added via a syringe and after completing that addition, oleic acid (9 ml) would be added dropwise into the solution. After several minutes, a black magnetic gel would precipitate. This gel can be isolated by magnetic decantation. The magnetic gel should be washed twice with sonication in acetone to remove excess oleic acid and re-suspended in chloroform to make a final suspension of 10 mg/ml in chloroform.

### Example 2

### Preparation of Biodegradable Magnetic Particles with Drug: Zotarolimus

pLGA (0.5 g, 50:50, 3A, Lakeshore Biomaterials) and the drug, zotarolimus (50 mg) would be dissolved in chloroform (3 ml) and mixed with the magnetite suspension in chloroform (5 ml) from Example 1. The chloroform mixture would then be added to a 2.5% solution of PVA (25 ml, M.W 9000-13000, Sigma-Aldrich). The resulted suspension would then be sonicated for ten minutes with a probe sonicator (Ultra Sonic, model CV18) at 100% power. The suspension would then be poured into stirred DI water (250 ml) and the suspension stirred overnight. The suspension would then be centrifuged at 16,000 rpm. After which, the particles would be re-suspended in DI water and centrifuged again until the supernatant is clear (3-4 times). Freeze-drying the particles would yield 0.45 gram of the desired particles. The size of the particles as measured on a Brookhaven ZetaPALS particle-sizing instrument would be expected to have an effective diameter of 198 nm with polydispersity of 0.083.

### Prophetic Example 1

### Preparation of Biodegradable Magnetic Particles with Drug: Everolimus

pLGA (0.5 g, 50:50, 3A, Lakeshore Biomaterials) and the drug, everolimus (50 mg) would be dissolved in chloroform (3 ml) and mixed with the magnetite suspension in chloroform (5 ml) from Example 1. The chloroform mixture would then be added to a 2.5% solution of PVA (25 ml, M.W 9000-13000, Sigma-Aldrich). The resulted suspension would then be sonicated for ten minutes with a probe sonicator (Ultra Sonic, model CV18) at 100% power. The suspension would then be poured into stirred DI water (250 ml) and the suspension stirred overnight. The suspension would then be centrifuged at 16,000 rpm. After which, the particles would be re-suspended in DI water and centrifuged again until the supernatant is clear (3-4 times). Freeze-drying the particles would yield 0.45 gram of the desired particles. The size of the particles as measured on a Brookhaven ZetaPALS particle-sizing instrument would be expected to have an effective diameter of 198 nm with polydispersity of 0.083.

### Prophetic Example 2

### Preparation of Biodegradable Magnetic Particles with Drug: Rapamycin

pLGA (0.5 g, 50:50, 3A, Lakeshore Biomaterials) and the drug, rapamycin (50 mg) would be dissolved in chloroform (3 ml) and mixed with the magnetite suspension in chloroform (5 ml) from Example 1. The chloroform mixture would then be added to a 2.5% solution of PVA (25 ml, M.W 9000-13000, Sigma-Aldrich). The resulted suspension would then be sonicated for ten minutes with a probe sonicator (Ultra Sonic, model CV18) at 100% power. The suspension would then be poured into stirred DI water (250 ml) and the suspension stirred overnight. The suspension would then be centrifuged at 16,000 rpm. After which, the particles would be re-suspended in DI water and centrifuged again until the supernatant is clear (3-4 times). Freeze-drying the particles would yield 0.45 gram of the desired particles. The size of the particles as measured on a Brookhaven ZetaPALS particle-sizing instrument would be expected to have an effective diameter of 198 nm with polydispersity of 0.083.

### Delivery of the Magnetic Particles and Localization of the Magnetic Particles in Rabbit Femoral Arteries

### Prophetic Example 3

### Zotarolimus

Twelve New Zealand White rabbits (2000-2500 g body weight, 12-15 weeks old) could be selected and divided into two groups of six each. One group would receive the magnetic particles from Example 2, while the other group would receive non-magnetic, biodegradable particles with zotarolimus.

The animals would be anesthetized with ketamine (35 mg/Kg) and xylazine (5 mg/Kg), and the femoral artery cannulized. A catheter containing a double-occluded balloon (Genie, Acrostak Inc., Germany) could be introduced. An electromagnet with a magnetic flux density of 1.7 Tesla could produce the magnetic field. The magnetic field would be focused onto the region between the double balloons with a pole placed in contact with the skin surface during magnetic particle perfusion. The first group of animals would be perfused with the magnetic particles encapsulating zotarolimus (20 mg/ml, 2 ml) in the balloon's occluded area while the magnetic field was activated. The double-occluded balloon would remain inflated for 10 minutes and then be withdrawn while the magnetic field would remain active for 120 minutes. To the second group the same procedure could be applied except using non-magnetic particles (20 mg/ml, 2 ml).

Afterward, the animals would be killed and the femoral arteries extracted. HPLC could be used to quantify the amount of zotarolimus in the arteries. This would show that arteries from the animals dosed with magnetic particles would have higher zotarolimus concentrations than the group dosed with non-magnetic particles.

### Prophetic Example 4

### Everolimus

Twelve New Zealand White rabbits (2000-2500 g body weight, 12-15 weeks old) could be selected and divided into two groups of six each. One group would receive the magnetic particles from prophetic example 1, while the other group would receive non-magnetic, biodegradable particles with Everolimus.

The animals would be anesthetized with ketamine (35 mg/Kg) and xylazine (5 mg/Kg), and the femoral artery cannulized. A catheter containing a double-occluded balloon (Genie, Acrostak Inc., Germany) could be introduced. An electromagnet with a magnetic flux density of 1.7 Tesla could produce the magnetic field. The magnetic field would be focused onto the region between the double balloons with a pole placed in contact with the skin surface during magnetic particle perfusion. The first group of animals would be perfused with the magnetic particles encapsulating everolimus (20 mg/ml, 2 ml) in the balloon's occluded area while the magnetic field was activated. The double-occluded balloon would remain inflated for 10 minutes and then be withdrawn while the magnetic field would remain active for 120 minutes. To the second group the same procedure could be applied except using non-magnetic particles (20 mg/ml, 2 ml).

Afterward, the animals would be killed and the femoral arteries extracted. HPLC could be used to quantify the amount of everolimus in the arteries. This would show that arteries from the animals dosed with magnetic particles would have higher everolimus concentrations than the group dosed with non-magnetic particles.

### Prophetic Example 5

### Rapamycin

Twelve New Zealand White rabbits (2000-2500 g body weight, 12-15 weeks old) could be selected and divided into two groups of six each. One group would receive the magnetic particles from prophetic example 2, while the other group would receive non-magnetic, biodegradable particles with Rapamycin.

The animals would be anesthetized with ketamine (35 mg/Kg) and xylazine (5 mg/Kg), and the femoral artery cannulized. A catheter containing a double-occluded balloon (Genie, Acrostak Inc., Germany) could be introduced. An electromagnet with a magnetic flux density of 1.7 Tesla could produce the magnetic field. The magnetic field would be focused onto the region between the double balloons with a pole placed in contact with the skin surface during magnetic particle perfusion. The first group of animals would be perfused with the magnetic particles encapsulating rapamycin (20 mg/ml, 2 ml) in the balloon's occluded area while the magnetic field was activated. The double-occluded balloon would remain inflated for 10 minutes and then be withdrawn while the magnetic field would remain active for 120 minutes. To the second group the same procedure could be applied except using non-magnetic particles (20 mg/ml, 2 ml).

Afterward, the animals would be killed and the femoral arteries extracted. HPLC could be used to quantify the amount of rapamycin in the arteries. This would show that arteries from the animals dosed with magnetic particles would have higher rapamycin concentrations than the group dosed with non-magnetic particles.

While particular embodiments of the present invention have been shown and described, it will be obvious to those skilled in the art that changes and modifications can be made without departing from the embodiments of this invention in its broader aspects and, therefore, the appended claims are to encompass within their scope all such changes and modifications as fall within the true, intended, explained, disclosed, and understood scope and spirit of this invention's multitudinous embodiments and alternative descriptions.

Additionally, various embodiments have been described above. For convenience's sake, combinations of aspects composing invention embodiments have been listed in such a way that one of ordinary skill in the art may read them exclusive of each other when they are not necessarily intended to be exclusive. But a recitation of an aspect for one embodiment is meant to disclose its use in all embodiments in which that aspect can be incorporated without undue experimentation. In like manner, a recitation of an aspect as composing part of an embodiment is a tacit recognition that a supplementary embodiment exists that specifically excludes that aspect. All patents, test procedures, and other documents cited in this specification are fully incorporated by reference to the extent that this material is consistent with this specification and for all jurisdictions in which such incorporation is permitted.

Moreover, some embodiments recite ranges. When this is done, it is meant to disclose the ranges as a range, and to disclose each and every point within the range, including end points. For those embodiments that disclose a specific value or condition for an aspect, supplementary embodiments exist that are otherwise identical, but that specifically exclude the value or the conditions for the aspect.

Finally, headings are for the convenience of the reader and do not alter the meaning or content of the disclosure or the scope of the claims.

What is claimed is:

1. A composition of matter comprising a drug and a magnetically sensitive drug carrier, wherein the composition is adapted for delivery to a mammal and capable of responding to a magnetic field.

2. The composition of matter of claim 1 wherein the composition takes the form of particles and wherein responding to a magnetic field means that the magnetically sensitive drug carrier particles experience a change in motion when exposed to the magnetic field.

3. The composition of claim 1 wherein causing a change in the motion of the magnetically sensitive drug carrier particles means changing the direction of the magnetically sensitive drug carrier particles or changing the velocity of the magnetically sensitive drug carrier particles or both.

4. The composition of matter of claim 2 wherein adapted for delivery comprises adapted for delivery by a delivery pathway or delivery route including a topical, enteral, or parenteral pathway or delivery route.

5. The composition of matter of claim 2 wherein the magnetically sensitive drug carrier is a nanoparticle, microparticle, liposome, micelle, nanofiber, or hydrogel.

6. The composition of matter of claim 1 wherein the magnetically sensitive drug carrier is a nanoparticle, microparticle, liposome, micelle, nanofiber, or hydrogel.

7. The composition of matter of claim 2 wherein the magnetically sensitive drug carrier comprises ferrite particles, ferrous oxide, or rare earth particles.

8. The composition of matter of claim 5 wherein the magnetically sensitive drug carrier responds to an external magnetic field

9. The composition of matter of claim 5 wherein the magnetically sensitive drug carrier responds to an internal magnetic field.

10. The composition of matter of claim 5 wherein the magnetic sensitive drug carrier comprises a material that exhibits a ferromagnetic, superparamagnetic, or paramagnetic effect.

11. The composition of matter of claim 10 wherein the drug inhibits the migration or proliferation of smooth muscle cells.

12. The composition of matter of claim 1 wherein the magnetically sensitive drug carrier responds to a magnetic field wherein responds to a magnetic field comprises the magnetically sensitive drug carrier's motion being changed by the magnetic field, the magnetically sensitive drug carrier's motion being changed such that the drug delivery is improved in any way over the same drug carrier absent the magnetic field source, the magnetically sensitive drug carrier's being directed to the desired treatment area long enough to improve or increase the drug transfer from the drug carrier to the tissue versus the drug carrier in the absence of the magnetic field and wherein delivery to a mammal comprises delivery to a coronary artery of a human patient.

13. A method comprising
administering a composition to a mammal, the composition comprising a magnetically sensitive drug carrier and a drug;
applying a magnetic field or magnetic field gradient along a preselected direction relative to a preselected target within the mammal.

14. The method of claim 13 wherein the step of administering is by oral, gastric feeding tube, duodenal feeding tube, gastrostomy, rectal, intravenous, intra-arterial, intramuscular, intracardial, subcutaneous, intraosseous infusion, intradermal, intrathecal, intraperitoneal, intravesical infusion, transdermal, transmucosal, sublingual, buccal, inhalational, epidural, or intravitreal pathway.

15. The method of claim 13 wherein capable of responding includes responding such that the rate that the particles move through the vessel inside of the magnetic field is lower than the rate that the particles move through the same or similar vessels absent the magnetic field by 10% or more; by 50% or more; by 80% or more; by 90% or more; by 95% or more; or by 99% or more.

16. The method of claim 13 wherein capable of responding includes responding such that the rate of transfer through the vessel is slower than the rate of diffusion of the drug from the particle by 10, 50, 60, 70, 80, 90, or 99%.

17. The method of claim 13 wherein the magnetic field is incorporated in a stent.

18. The method of claim 13 further comprising a step of waiting a preselected period after the step of administering a composition to a mammal before applying a magnetic field or magnetic field gradient along a preselected direction relative to preselect target.

19. The method of claim 18 wherein the preselected period is from 0 to 24 hours.

20. The method of claim 19 wherein the preselected period is from 0 to 1 hour.
